(19) United States
(12) Patent Application Publication
(10) Pub. No.: US 2013/0053270 A1
(43) Pub. Date: Feb. 28, 2013

(54) METHODS FOR DETERMINING GENE-NUTRIENT INTERACTIONS

(71) Applicants: Rosalynn D. Gill, Boulder, CO (US); Keith A. Grimaldi, Sorrento (IT)

(72) Inventors: Rosalynn D. Gill, Boulder, CO (US); Keith A. Grimaldi, Sorrento (IT)

(73) Assignee: BODYSYNC, INC., Redondo Beach, CA (US)

(21) Appl. No.: 13/580,604

(22) PCT Filed: Feb. 24, 2011
(86) PCT No.: PCT/US11/26108

§ 371 Date: Nov. 6, 2012

Related U.S. Application Data
(60) Provisional application No. 61/307,526, filed on Feb. 24, 2010.
(60) Provisional application No. 61/307,522, filed on Feb. 24, 2010.

(51) Int. Cl. C12Q 1/68; C40B 30/04

(52) U.S. Cl. 506/9; 435/6.11

(57) ABSTRACT

The present invention provides methods and tests that allow for the establishment of personalized weight-management programs for an individual based upon the individual's genotype in the glutathione S-transferase pi gene and/or the interleukin-6 gene. Methods are disclosed for determining the individual's genotype, which may be used to select an appropriate therapeutic/dietary program or lifestyle recommendation. Such a personalized weight-management program will have obvious benefits (e.g., yield better results in terms of weight loss and weight maintenance) over traditional weight-management programs that do not take into account genetic information.

### FIELD OF THE INVENTION

The present invention relates to methods for predicting metabolic responses to dietary factors and to providing dietary and lifestyle advice based on gene-nutrient interactions, based on polymorphisms in the glutathione S-transferase pi gene (GSTP1) and/or the interleukin-6 gene (IL-6).

### BACKGROUND TO THE INVENTION

There has been a considerable change in the dietary habits of people living in industrial regions since the second half of the twentieth century. Traditional diets that were largely plant based have been replaced by diets that are high in fat or calories and have a substantial content of animal based foods. There has also been a general decrease in physical activity in industrial countries as a result of a progressive shift toward a more sedentary lifestyle.

Diet and physical inactivity are known to be key risk factors associated with weight gain or the development of obesity. The number of people being clinically diagnosed as overweight or obese is increasing and for many health advisory bodies, excessive weight gain and obesity is now an important health concern. Obesity is a chronic disease that affects all age groups and is associated with a number of health risks, such as high blood pressure, coronary heart disease or diabetes. A person is obese if they have excessive bodily fat and/or if they are extremely overweight i.e. their weight is significantly greater than what is considered generally healthy for a person of that age, height and nationality. The presence of excessive bodily fat also increases the risk that the person will suffer from a physical disability.

For many decades, Governments, charities and health advisory bodies have issued health advice, for example relating to diet, exercise, smoking and sunbathing. The dietary or exercise guidelines provided by such organisations aimed at preventing weight gain or obesity tends to be directed at the public as a whole, or, at best, to groups such as the elderly, children and pregnant women. This advice can therefore only be very general and cannot, by its very nature, take into account personalised risk factors, such as the genetic characteristics of an individual. Moreover, in recent years, research findings on links between particular foods, drugs etc and medical conditions, have received large amounts of publicity, often causing health scares.

The factors that contribute to health status and susceptibility to medical conditions vary between populations and between individuals within populations, so it is often impossible for an individual to derive useful advice appropriate to his or her particular circumstances from such general reports and research.

In order for physicians and other professionals to provide advice more tailored to an individual's needs, it would be desirable to analyse the individual's genetic make up to identify any genes associated with positive or negative effects. By determining the mechanism of the effects of nutrients or the effects of a nutritional regime, the science of nutrigenetics (also referred to as nutrigenomics) tries to define the relationship between these specific nutrients and specific nutrient regimes on human health. The technology for examining multiple gene variations in an individual's genome is currently well advanced. The current problem in the field of nutrigenetics is to identify a sufficiently large pool of variations such that meaningful advice can be provided to individuals in a manner that will contribute to their health and fitness.

### SUMMARY OF INVENTION

The present invention includes a method for selecting a weight loss program for an individual. In one aspect the method comprises determining the individual's GSTP1 genotype at loci position 313, 341 or both and selecting a weight loss program for the individual when the individual comprises a genotype selected from the group consisting of homozygous for the G allele at position 313, heterozygous (A/G) at position 313, homozygous for the T allele at position 341, heterozygous (C/T) at position 341, and combinations thereof wherein the weight loss program is modified from a weight loss program for a comparable individual who is wild-type at positions 313 and 341. This method can further provide when the genotype of the individual comprises either homozygous for the G allele at position 313 or heterozygous (A/G) at position 313, the individual is predicted to obtain a greater response to the weight loss program compared to an individual homozygous for the A allele at position 313. Another aspect of this method of the present invention further provides when the genotype of the individual comprises either homozygous for the T allele at position 341 or heterozygous (C/T) at position 341, the individual is predicted to obtain a greater response to the weight loss program compared to an individual homozygous for the C allele at position 341.

In another aspect of the present invention, the method further provides when the genotype of the individual comprises either homozygous for the G allele at position 313 or heterozygous (A/G) at position 313, the selected weight loss program comprises a dietary program higher in energy intake or shorter in duration compared to a weight loss program for a comparable individual having a wild-type genotype at position 313. This method can further comprises the selected weight loss program comprising a higher calcium intake compared to a weight loss program for a comparable individual having a wild-type genotype at position 313.

In still another aspect of the present invention, the method further provides when the genotype of the individual is homozygous for the C allele at position 341, a weight loss program comprising a dietary program lower in energy intake or longer in duration compared to an individual having a genotype consisting of a T allele at position 341.

In another aspect of the present invention, the method further provides when the genotype of the individual is homozygous for the A allele at position 313 and/or homozygous for the C allele at position 341, a weight loss program comprising a dietary program lower in energy intake or longer in duration compared to an individual having a genotype comprising homozygous for the G allele at position 313 or heterozygous (A/G) at position 313 and comprising homozygous for the T allele at position 341 or heterozygous (C/T) at position 341.

In yet another aspect of the present invention, the method further provides when the genotype of the individual is either homozygous for the T allele at position 341 or heterozygous (C/T) at position 341, a selected weight loss program comprising a dietary program higher in cruciferous vegetable intake compared to a weight loss program for a comparable individual having a wild-type genotype at position 341.

In still another aspect of the present invention, the method further provides when the genotype of the individual is either homozygous for the G allele at position 313 or heterozygous (A/G) at position 313, a selected weight loss program comprising a dietary program with higher vitamin A intake compared to a weight loss program for a comparable individual having a wild-type genotype at position 313.

In another aspect of the present invention, the method further provides when the genotype of the individual is either homozygous for the G allele at position 313 or heterozygous (A/G) at position 313, a selected weight loss program comprising a dietary program with higher calcium intake compared to a weight loss program for a comparable individual having a wild-type genotype at position 313.

In yet another aspect of the present invention, the method further provides when the genotype of the individual is either homozygous for the G allele at position 313 or heterozygous (A/G) at position 313 and is either homozygous for the T allele at position 341 or heterozygous (C/T) at position 341, a selected weight loss program comprising a dietary program with higher vitamin A intake compared to a weight loss program for a comparable individual having a wild-type genotype at position 313 and with higher cruciferous vegetable intake compared to a weight loss program for a comparable individual having a wild-type genotype at position 341.

In still another aspect of the present invention, the method further provides when the genotype of the individual comprises a genotype of homozygous for the G allele at position 313 or heterozygous (A/G) at position 313 and comprises homozygous for the T allele at position 341 or heterozygous (C/T) at position 341, a selected weight loss program comprising a dietary program with higher calcium intake compared to a weight loss program for a comparable individual having a wild-type genotype at position 313 and comprises a dietary program with higher cruciferous vegetable intake compared to a weight loss program for a comparable individual having a wild-type genotype at position 341.

The present invention also provides a method for selecting a dietary plan for an individual in need of achieving an increase in body weight. This method comprises determining the individual's GSTP1 genotype at loci position 313 of the GSTP1 gene and selecting a dietary program for the individual when the individual is homozygous for the A allele at position 313, wherein the dietary program recommends a lower calcium intake compared to a dietary program for an individual who is not in need of achieving an increase in body weight.

Another aspect of the present invention provides that the GSTP1 allele is determined as part of a panel of at least 5 genes that have one or more alleles. This method further provides that the other genes of the panel are selected from methylene-metra-hydro-folate-reductase (MTHFR); metyhionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein C-III (APOC3); apolipoprotein A-V (APOA5); cholesteryl ester transfer protein (CETP); ipoprotein lipase (LPL); endothelial nitric oxide synthase (eNOS); angiotensin converting enzyme gene (ACE); vitamin D receptor (VDR); collagen type I alpha 1 (COL1A1); tumor necrosis factor alpha (TNF-α); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

The present invention also provides for a method for selecting a weight loss program for an individual under the age of 50. This method further comprises determining the individual's IL-6 genotype at loci position −174. The method further comprises selecting a weight loss program for the individual when the individual comprises a genotype selected from the group consisting of homozygous for the G allele at position −174 and heterozygous (C/G) at position −174, wherein the weight loss program is modified from a weight loss program for a comparable individual who is homozygous for the C allele at position −174. This method further provides when the individual's genotype is homozygous for the C allele at position −174, the individual is predicted to obtain a greater response to a weight loss program compared to an individual having a genotype selected from the group consisting of homozygous for the G allele at position −174 and heterozygous (C/G) at position −174. In yet another aspect of this method of the present invention, the method further provides when the individual's genotype is selected from the group consisting of homozygous for the G allele at position −174 and heterozygous (C/G) at position −174, the selected weight loss program comprises a dietary program lower in energy intake and/or longer in duration compared to an individual comprising a genotype of homozygous for the C allele at position −174. In still another aspect of this method, the method further provides that the IL-6 allele is determined as part of a panel of at least 5 genes that have one or more alleles. This method further provides that the other genes of the panel are selected from methylene-metra-hydro-folate-reductase (MTHFR); metyhionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein C-III (APOC3); apolipoprotein A-V (APOA5); cholesteryl ester transfer protein (CETP); ipoprotein lipase (LPL); endothelial nitric oxide synthase (eNOS); angiotensin converting enzyme gene (ACE); vitamin D receptor (VDR); collagen type I alpha 1 (COL1A1); tumor necrosis factor alpha (TNF-α); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

### DETAILED DESCRIPTION OF THE INVENTION

The present inventors have identified associations between two alleles of GSTP1 and body mass index, and further found relationships between certain dietary factors and these alleles. In addition, the present inventors have identified associations between the −174 alleles of IL-6 and body mass index associated with age. By assessing whether or not these alleles are present in individuals, it is possible to select weight-management programs for individuals.

The present invention relates to methods for selecting a weight management program, such as a weight loss program, for an individual by determining the individuals GSTP1 genotype at loci position 313, 341 or both.

The glutathione S-transferase pi gene (GSTP1) is a polymorphic gene encoding active, functionally different GSTP1 variant proteins that are thought to function in xenobiotic metabolism and play a role in susceptibility to cancer, and other diseases. However, no associations with normal dietary factors have been reported to date. The sequence of the GSTP1 gene (open reading frame) and translation thereof is shown as SEQ ID NO:1 and SEQ ID NO:2 respectively. The wild-type (cDNA) sequence is shown (SEQ ID NO:3). The numbering is based on the open reading frame, with the first methionine ATG being numbered 1-3 of the sequence.

There are two common allelic variants of GSTP1. One is at position 313 of the open reading frame of the nucleic acid, which changes A to G (SEQ ID NO:4), the other at position 341 is a C to T change (SEQ ID NO:6). Both changes also cause a change to the coding sequence, resulting in the protein variants Ile105Val (SEQ ID NO:5) and Ala114Val (SEQ ID NO:7). The gene is autosomal; thus individuals can be homozygous or heterozygous at each allele.

The sequence of the GSTP1 gene (open reading frame) and translation thereof is shown as SEQ ID NO:1 and SEQ ID NO:2 respectively. The wild-type (cDNA) sequence is shown (SEQ ID NO:3). The numbering is based on the open reading frame, with the first methionine ATG being numbered 1-3 of the sequence. As indicated above, the changes at positions 313 and 341 also give rise to coding sequences and are also referred to in the literature as Ile105Val and Ala114Val respectively. In this invention the alleles are referred to by reference to the nucleotide numbering and changes, since genetic screening is primarily done by reference to nucleotide analysis.

Single nucleotide polymorphisms are also classified by the Database of Single Nucleotide Polymorphisms (dbSNP), Bethesda (MD): National Center for Biotechnology Information, National Library of Medicine (see Sherry S T, et al; dbSNP: the NCBI database of genetic variation. Nucleic Acids Res. 2001 Jan. 1; 29(1):308-11). The SNPs are catalogued by unique accession numbers. In the present case, the GSTP1 A313G polymorphism is SNP accession number rs1695 and the GSTP1 C341T polymorphism is rs1138272.

An embodiment of the invention includes determining an individual's GSPT1 genotype at loci position 313, 341 or both. The genotype of an individual will generally be determined by analysis of a sample of nucleic acid, normally DNA, obtained from the individual, e.g. in the form of a buccal swab or similar sample. The analysis will take place using conventional methods known as such in the art. This may include use of PCR to amplify and sequence the gene at one or both positions 313 and 341 or the use of nucleic acid probes that are capable of distinguishing between the alleles by differentially hybridizing to the wild-type and variant sequences. Since the alleles are also reflected in protein coding changes it is possible that the alleles may be detected at the protein level, e.g. by immunoassay or other protein analytical methods. Such methods would be practiced using a sample from the individual which contains detectable levels of GSTP1 protein.

This embodiment of the present invention also includes when the genotype of the individual is either homozygous for the G allele at position 313 or heterozygous (A/G) at position 313, the individual is predicted to obtain a greater response to the weight loss program compared to an individual homozygous for the A allele at position 313. In addition, when the individual's genotype is determined to be homozygous for the T allel at position 341 or heterozygous (C/T) at position 341 the individual is also predicted to obtain a greater response to the weight loss program compared to an individual homozygous for the C allele at position 341.

Another embodiment of the present invention includes when the individual's genotype is either homozygous for the G allele at position 313 or heterozygous (A/G) at position 313, the selected weight loss program comprises a dietary program higher in energy intake or short in duration compared to weight loss program for a comparable individual having a wild-type genotype at position 313. This method further provides selecting a weight loss program comprising a higher calcium intake compared to a weight loss program for a comparable individual having a wild-type genotype at position 311.

There is an association between BMI and calcium intake in subjects with different alleles of the GSTP1 313 polymorphism. Those homozygous for the AA allele appear to benefit from a low calcium intake, wherein those homozygous for the G allele at position 313 or heterozygous (A/G) at position 313 benefit from a high intake. By a low intake, it is meant the intake may be up to 1000 mg per day, for example up to 900 mg per day, such as up to 800 mg per day. By a high intake, it is meant the intake may be at least 1100 mg per day, for example at least 1200 mg per day, such as 1300 mg per day.

In some embodiments of the present invention the method further provides when the genotype of the individual comprises either homozygous for the T allele at position 341 or heterozygous (C/T) at position 341, a weight loss program comprising a dietary program higher in cruciferous vegetable intake compared to a weight loss program for comparable individual having a wild-type genotype at position 341.

Edible plants in the family Brassicaceae (also called Cruciferae) are termed cruciferous vegetables. The most commonly eaten of such vegetables include cabbage, broccoli, cauliflower, kale, Brussels sprouts, turnip, rapeseed, mustard, radish, horseradish, cress and watercress. Using the methods of the present invention individuals having a GSTP1 T allele (i.e. a heterozygous CT or homozygous TT genotype) at position 341 with a high intake of cruciferous vegetables have a positive association with a lower BMI compared to CC homozygotes. Suitably, a minimum level of cruciferous vegetable intake associated with the benefit observed in CT or TT genotypes may be at least 3 servings of cruciferous vegetables per week, for example at least 5 servings per week, such as at least 7 servings per week. A serving of cruciferous vegetables is considered to be a portion of approximately 100 g of the vegetable.

In some embodiments of the present invention the method further provides when the genotype of the individual comprises either homozygous for the G allele at position 313 or heterozygous (A/G) at position 313, a selected weight loss program comprising a dietary program with higher vitamin A intake compared to a weight loss program for comparable individual having a wild-type genotype at position 313.

Vitamin A intake in an individual may come from a combination of foodstuffs and in the form of vitamin supplements, generally in the form of retinol or carotenes which are converted into retinol in the body. Individuals homozygous for the G allele at position 313 or heterozygous (A/G) at position 313 there is a higher BMI associated with a low intake of the vitamin. Thus such individuals may benefit from increasing vitamin A intake in their diet, either by increasing food sources with this vitamin and/or by taking vitamin A supplements. Suitably, a minimum level of Vitamin A intake associated with the benefit observed in AG and GG genotypes may be at least 3000 (international units) per day, for example at least 4000 IU per day, such as at least 5000 IU per day.

In this and the other methods of the invention, the recommendations of levels of intake of food subtypes (i.e., as the case may be, cruciferous vegetables, vitamin A or calcium) may be modified taking into account questionnaire data relating to current diet. For example, where an individual likely to benefit from a high cruciferous vegetable intake is already exceeding the minimum intake, the recommendation of a minimum level may be that such a level will be achieved with the current dietary intake.

A further embodiment of the present invention is a method for selecting a dietary plan for an individual in need of achieving an increase in body weight (i.e. a weight-gain program). This method comprises determining the individual's GSPT1 genotype at loci position 313 of the GSPT1 gene. In particular, since individuals with a 313 A homozygous geneotype with a high-calcium diet have a higher BMI than those with a G allele, such subjects could be prescribed a high-calcium dietary supplement, or recommended a diet rich in calcium, to assist in achieving a weight gain target. Individuals homozygous for the A allele at position 313 could be prescribed a lower calcium intake dietary program compared to an individual who is not in need of achieving an increase in body weight. In addition, the present invention also contemplates determining the GSPT1 genotype at position 341 of the GSPT1 gene for selecting a dietary plan for an individual in need of achieving an increase in body weight.

Another embodiment of the present invention is a method for selecting a weight management program, such as a weight loss program, for an individual by determining the individuals IL-6 genotype at loci position −174. The interleukin-6 (IL-6) gene (SEQ ID NO:10 represents wild-type IL-6 which encodes SEQ ID NO:11) is a polymorphic gene in which the nucleotide sequence in the promoter region at position −174 may be C or G (SEQ ID NO: 8). Single nucleotide polymorphisms are classified by the Database of Single Nucleotide Polymorphisms (dbSNP), Bethesda (MD): National Center for Biotechnology Information, National Library of Medicine (see Sherry S T, et al; dbSNP: the NCBI database of genetic variation. Nucleic Acids Res. 2001 Jan. 1; 29(1):308-11). The SNPs are catalogued by unique accession numbers. In the present case, the IL-6-174 polymorphism is accession number rs1800795 (SEQ ID NO:9).

The genotype of an individual will generally be determined by analysis of a sample of nucleic acid, normally DNA, obtained from the individual, e.g. in the form of a buccal swab or similar sample. The analysis will take place using conventional methods known as such in the art. This may include use of PCR to amplify and sequence the gene at position −174 or the use of nucleic acid probes that are capable of distinguishing between the alleles by differentially hybridizing to the wild-type and variant sequences.

In this embodiment a method for selecting a weight loss program for an individual under the age of 50 is provided. This method comprises determine the individuals IL-6 genotype at loci position −174 and selecting the weight loss program when the individual is either homozygous for the G allele at position −174 or heterozygous (C/G) at position −174 where the weight loss program is modified from a weight loss program for a comparable individual who is wild-type at position −174.

In many embodiments of the invention, the alleles of GSTP1 and/or IL-6 will be determined in a gene chip array in which a number of other gene variants associated with lifestyle and dietary risk factors are also analysed.

Although the invention may be performed by examining the GSTP1 alleles and/or the IL-6 allele in isolation, it is also contemplated that the alleles will be determined as part of a panel of genes associated with diet and health.

It is also contemplated that where the individual's genotype is such that two or more of the dietary factors mentioned above are associated with beneficial or detrimental effects, the invention provides for a lifestyle dietary advice plan relating to any combination of such factors.

### “Individual” or “Subject”

The invention is intended for performance on human subjects. Generally the human will be adult, i.e. age 18 or above. The subjects may be men or women.

The associations reported herein were determined in Caucasian subjects of both sexes, with no significant differences being identified between men and women. However, unlike haplotype analysis, where linkage disequilibrium may occur in different ethnic subpopulations, the present invention relates to alleles in protein coding regions. This indicates that the differences between different genotypes are a result of the change to the structure and hence activity of the GSTP1 protein. Accordingly the invention may also be practiced on subjects of other ethnic population groups, e.g. those of black African or oriental origin, as the activity of the protein will be similar in all population groups, regardless of differences in the particular frequency of the particular alleles.

In regards to the polymorphism in IL-6, the associations reported herein were determined in Caucasian subjects of both sexes, with no significant differences being identified between men and women. It is believed that the polymorphism in IL-6 affects the expression levels of the gene, and that this difference will be maintained in all ethnic subgroups, even where the frequencies of the alleles vary. Thus, the invention may also be practiced on subjects of other ethnic population groups, e.g. those of black African or oriental origin, as the activity of the gene will be similar in all population groups, regardless of differences in the particular frequency of the particular alleles.

In the present invention a comparable individual is an individual or group of individuals that have been determined to have wild-type GSPT1 313 (homozygous for the A allele), 341 (homozygous for the T allele) and/or IL-6-174 alleles (homozygous for the C allele) as the case may be for the relevant method of the present invention. In addition, comparable individuals are similar to the individual who is the subject of the method in other relevant characteristics for the method. For example, such relevant characteristics can include age, weight, height, health history, sex, other genetic characteristics, lifestyle factors and/or diet.

### Predicting the Response to a Weight Loss Program.

The GSTP1 or the IL-6 polymorphisms of the present invention may be used by individuals or heath care practitioners to predict responses to a weight loss program. The data of the present invention show that on a balanced calorie controlled diet, the weight loss of subjects with a GSTP1 313 G or a GSTP1 341 T allele was greater than in subjects with wild-type alleles. In addition, the data of the present invention show that there is both an age-related and IL-6 allele-related effect on BMI with the IL-6 gene.

This information may be used to advise individuals on such diets who carry the wild-type alleles that their weight loss may be smaller than those with variant alleles. Such information may be useful in for example weight-loss programs in clinical settings or in profit-oriented or non-profit weight loss organizations. The exact degree of weight loss will depend on the nature of the diet and/or exercise regime to accompany the diet. For each program the historic or predicted average weight loss of participants can be modified for an individual in the light of the GSTP1 or IL-6 genotype such that expectations or targets of that individual are more tailored to their particular genetic make up. For example, individuals with GSTP1 wild-type alleles may be advised that their weight loss is likely to be less than average for participants following the same diet or exercise program, whereas those with a GSTP1 313 G or a GSTP1 341 T allele can be predicted to lose more than average over the course of a similar program. In addition, individuals under age 50 with a IL-6 G allele (“higher BMI-associated profile”) may be advised that their weight loss is likely to be less than average for participants following the same diet or exercise program, whereas those not with these age-genotype combinations (“lower BMI-associated profile”) can be predicted to lose more than average over the course of a similar program.

### Dietary Program

As well as predicting the relative likely weight loss on a particular diet, the invention may also allow, within the context of such programs, the opportunity to tailor such programs based on the GSTP1 genotype or the IL-6 genotype. For example, individuals with wild-type GSTP1 alleles could be counseled not only in their expectations of likely weight loss, but also given guidance and advice on means to achieve greater loss by either lowering the calorific intake of the diet, increasing exercise, or participating in the program for longer. Similarly, those with a GSPT1 313 G or a GSPT1 341 T allele could be set a more ambitious weight-loss target, or the dietary program modified to allow a higher calorie diet than those with the wild-type allele.

Precise energy intake for a diet for an individual will need to be determined by the individual concerned, where necessary or appropriate in consultation with a health-care advisor, and thus precise numbers are unlikely to be applicable to all individuals in all circumstances. Generally, the calorific intake of an individual on a diet is in the range of 1000-2000 kilocalories/day (approx 4200-8400 kJ/day). Thus it may be that for a group of individuals participating in a particular weight loss regime, such a group could be divided according to the BMI-associated profiles of the invention and those with the higher BMI-associated profile (i.e. homozygous for the A allele at position 313 or homozygous for the C allele at position 341) prescribed a diet which is reduced by, e.g. about 5-20, such as about 5-10% in energy intake than those individuals with a lower BMI-associated profile (individuals who have a G allele at position 313 or individuals who have a T allele at position 341).

In regards to the IL-6 genotype, individuals with a higher BMI-associated profile could be counseled not only in their expectations of likely weight loss, but also given guidance and advice on means to achieve greater loss by either lowering the calorific intake of the diet, increasing exercise, or participating in the program for longer. Similarly, those with a lower-BMI associated profile could be set a more ambitious weight-loss target, or the dietary program modified to allow a higher calorie diet than those with the wild-type allele. In addition, those with a lower-BMI associated profile could be advised to control their weight carefully while young in order to mitigate the increased risk of a higher BMI from the age of 50 onwards.

### Nutrigenetic Screening

The field of nutrigenetic screening involves the analysis of one or more genes in a subject which is involved in a response to a dietary or other health-associated factor, and in which one or more alleles that may alter that response have been identified. In a typical procedure, a sample of DNA from a subject is provided. This may be in the form of a buccal swab or other body sample. The DNA is then examined to determine which alleles of one or more genes of interest are present. Where alleles of genes which are identified that give rise to increased risk of one or more adverse outcomes (e.g. lower bone mineral density, higher risk of heart disease, etc) the individual may be advised to modify his or her diet by to account for that risk. For example, the advice may include recommended minimum and/or maximum amounts of food subtypes, such as fats, vegetable subgroups (brassicas, alliums, etc). Such a method may be the method of U.S. Pat. No. 7,054,758 referred to above.

In some embodiments of nutrigenetic screening, the individual may also provide, in conjunction with a DNA sample, a response to a questionnaire providing lifestyle details (for example such as one or more of current diet, age, sex, alcohol intake and whether or not they are a smoker). This can allow the advice to be further tailored to the requirements of the individual.

In a typical method of nutrigenetic screening, the alleles of GSTP1 may be determined within a panel of from 5 to 100, such as from 5 to 20 other genes which have allelic variants associated with responses to, or risk factors for, diet or health. The genes which may be included in the panel may be selected from methylene-metra-hydro-folate-reductase (MTHFR); metyhionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); interleukin-6 (IL-6); apolipoprotein A-V (APOA5); apolipoprotein C-III (APOC3); cholesteryl ester transfer protein (CETP); lipoprotein lipase (LPL); endothelial nitric oxide synthase (eNOS); angiotensin converting enzyme gene (ACE); vitamin D receptor (VDR); collagen type I alpha 1 (COL1A1); tumor necrosis factor alpha (TNF-α); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

Also, in a typical method of nutrigenetic screening, the alleles of IL-6 may be determined within a panel of from 5 to 100, such as from 5 to 20 other genes which have allelic variants associated with responses to, or risk factors for, diet or health. The genes which may be included in the panel may be selected from methylene-metra-hydro-folate-reductase (MTHFR); metyhionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein A-V (APOA5); apolipoprotein C-III (APOC3); cholesteryl ester transfer protein (CETP); lipoprotein lipase (LPL); endothelial nitric oxide synthase (eNOS); angiotensin converting enzyme gene (ACE); vitamin D receptor (VDR); collagen type I alpha 1 (COL1A1); tumor necrosis factor alpha (TNF-α); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

The polymorphisms of the gene panel for the above genes, when included in the panel, may be selected from the following genes listed in Table 1:

TABLE 1

| Gene | Genetic Variations | rs Number (where applicable) |
|---|---|---|
| MTHFR | C677T; | rs1801133 |
| | A1298C | rs1801131 |
| MS-MTRR | A66G | rs1801394 |
| MTR | A2756G | rs1805087 |
| CBS | C699T | rs234706 |
| MnSOD | C(−28)T | rs4880 |
| SOD3 | C760G | rs1799895 |
| GSTM1 | Present or Deleted | n/a |
| GSTT1 | Present or Deleted | n/a |
| APOA5 | T1131C; | rs662799 |
| | C56G | rs3135506 |
| IL-6 | G(−174)C; | rs1695 |
| | G(−634)C | rs1138272 |
| APOC3 | C3175G | rs5128 |
| CETP | G279A | rs708272 |
| LPL | C1595G | rs328 |
| eNOS | G894T | rs1799983 |
| ACE | Insertion/Deletion | rs4646994 |
| VDR | TBsmIC; | rs1544410 |
| | CTaqIT; | rs731236 |
| | TFokIC | rs10735810 |
| COL1A1 | GSp1T | rs1800012 |
| TNF-α | G(−308)A | rs1800629 |
| PPAR-γ2 | ProI2 AIA | rs1801282 |
| EPHX1 | Tyr113His | rs1051740 |
| LIPC | G250A; | rs2070895 |
| | C514T | rs1800588 |
| PON1 | Gln192Arg; | rs662 |
| | Leu55Met | rs854560 |
| ADH1B | Arg369Cys; | rs2066702 |
| | Arg47His | rs1229984 |
| ADH1C | Ile349Val | rs698 |
| AGT | Met235Thr | rs699 |
| CYP1A1 | A2455G | rs1048943 |
| CYP1A2_1B | C1548T | rs2470890 |
| CYP1A2_1E | G740GT | rs2069526 |
| CYP1A2_1F | A163C | rs762551 |
| GSTP1 | A131G | rs1695 |
| | C341T | rs1138272 |

Thus various the methods of the present invention described herein may be practiced either on the GSTP1 gene alone, to determine one or both of the alleles at 313 and 341, or as part of a nutrigenetic screening method. When the latter, the method may include the determination of an allele of one or more of the genes of the above Table.

In addition, various methods of the present invention described herein may be practiced either on the IL-6 gene alone, to determine the −174 allele, or as part of a nutrigenetic screening method. When the latter, the method may include the determination of an allele of one or more of the genes of Table 1.

### Body Mass Index (BMI)

It is commonly known in the art, that the terms overweight and obesity refer to ranges of weight that are greater than what is generally considered healthy for a given height. A person may be classified as being overweight or obese by using their weight measurement and height to calculate their body mass index (BMI). Body mass index may be calculated using the following formula:

BMI (kgm⁻²)=bodyweight (kg)÷[height (m)]².

BMI tends to be used to categorise individuals as being overweight or obese because, for most people, it correlates with their amount of body fat. For adults, a healthy BMI is typically between 18.5 and 25 kgm⁻². If the adult has a BMI of at least 25 kgm⁻², but less than 30 kgm⁻² they are typically classified as being overweight. A BMI greater than or equal to 30 kgm⁻² indicates that the adult is obese.

The present invention may be useful in underweight, healthy-weight (BMI of 18.5 to 25), overweight and obese individuals. The invention may be particularly useful in those classified as overweight or obese when used as part of a dietary program. The use of the invention in healthy-weight individuals may also be of use in providing advice in maintenance of a BMI in the 18.5-25 range.

Various methods of determining a personalised lifestyle advice plan for human subjects are disclosed in U.S. Pat. No. 7,054,758, the disclosure of which is incorporated herein by reference. In general, methods comprise the steps, which are usually computer-assisted, of:

(i) providing a first dataset on a data processing device, said first dataset comprising information correlating the presence of individual alleles known to be associated with increased or decreased disease susceptibility, with a lifestyle risk factor;

(ii) providing a second dataset on a data processing device, said second dataset comprising information matching each said risk factor with at least one lifestyle recommendation;

(iii) inputting a third dataset identifying alleles present in said subject, wherein said alleles are one or more of the alleles of said first dataset;

(iv) determining the risk factors associated with said alleles present in said human subject by correlating said alleles with risk factors provided by said first dataset;

(v) determining at least one lifestyle recommendation based on each identified risk factor from step (iv) by matching said risk factor with a lifestyle recommendation from said second dataset; and

(vi) generating a personalized lifestyle advice plan based comprising at least one lifestyle recommendation determined in step (v).

The personalized lifestyle advice plan may include recommended minimum and/or maximum amounts of food subtypes. The associations disclosed herein with the GSTP1 gene may be used to provide alleles for step (i) of the above process, risk factors relating to BMI and current dietary intake for step (ii) and the recommendations disclosed herein for step (v) in order to generate a personalised lifestyle advice plan which takes account of the GSTP1 genotype, amongst other genetic markers.

An additional embodiment of the invention provides a kit and a method of use of the kit, wherein the kit comprises means for collection of a DNA sample of an individual and optionally a questionnaire to gather data relating to the individuals BMI and intake of one or more of cruciferous vegetables, vitamin A and calcium. By “data relating to BMI” this may be the BMI itself or height and weight data allowing BMI to be calculated. The method of use of the kit will comprise the steps of analysing the DNA sample to determine the genotype of the individual at GSTP1 positions 313 and/or 341 and/or IL-6; and providing a recommendation relating to diet and/or lifestyle (e.g. exercise or activity levels) based on the determination of the GSTP1 genotype and/or the IL-6 genotype.

This application hereby incorporates by reference U.S. Application Nos. 61/307,522 and 61/307,526 in their entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

### EXAMPLES

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure.

As described below in examples 1-5, the response (weight loss) of individuals to a controlled diet was found to be greater in those who had one or other of the variant genotypes (AG or GG at position 313, CT or TT at 341) than those with the wild-type alleles. Individuals with a variant genotype (CT or TT) at 341 were found to have a lower Body Mass Index (BMI) than those with the wild-type alleles.

The data provided in examples 1-5 show that there is an association of BMI and weight regulation and the GSTP1 genotype. This association allows predictions to be made as to the likely outcomes to diet and exercise dependent upon genotype, and allows individuals, or health professionals advising such individuals, to make decisions about dietary and/or exercise programs likely to be of benefit in achieving weight loss or maintaining a healthy weight.

Accordingly, the findings of the present invention may be used to provide both general dietary and/or lifestyle advice based on the GSTP1 genotypes of individuals, as well as more specific advice on food subtype intake, i.e. in regard to one or more of cruciferous vegetables, vitamin A and calcium.

### Example 1

This example illustrates the genetic association between GSPT1 polymorphism and weight loss. 41 patients with a history of unsuccessful attempts at weight loss (defined as at least two or more unsuccessful attempts) attending a weight management clinic in Athens, Greece followed a traditional weight management program involving a low glycemic index Mediterranean diet, recommended exercise routines and regular follow-up visits in the clinic as follows:

BREAKFAST: One cup of coffee or tea, One thin slice of whole grain bread or rye biscuit with one slice of cheese and a slice of turkey ham or with margarine (Becel™) and little honey or one portion of cereal with low fat 1.5% milk

### Lunch-Dinner:

Day 1: One salad of fresh or boiled vegetables, one slice of cheese, one slice of bread.
Day 2: Grilled fish+salad
Day 3: Grilled Chicken+salad
Day 4: One portion of green beans, cooked with tomato & olive oil. One slice of cheese
Day 5: Grilled fillet+salad
Day 6: One portion of lentils, one slice of cheese, one slice of bread
Day 7: Grilled fish+salad
The dietary program of the patients was modified from the standard diet based on the genetic results of the GSTP1 variants at sites 313 or 341. Patients carrying one or two copies of the variant alleles at positions 313 or 341 of the Glutathione S-transferase pi gene were recommended to ensure that diet included regular portions of cruciferous (5 times per week) and allium (daily) vegetables with suggestions and recipes provided to the patient, and to add broccoli extract and allium supplement if required.

BMI test results were analyzed from patients' clinical records at regular intervals. 41 individuals had their BMI measured after 100 days (average follow-up 167 days or 5.6 months).

For nutrigenetic testing, the Sciona Body Benefits kit was used (Sciona Inc Boulder Colo.). Cheek cell samples were taken in the clinic using two buccal swabs and the patient completed a comprehensive diet and lifestyle questionnaire. The swabs and samples were sent by courier to Sciona and genetic testing was carried out using a Sequenom Mass Array system.

Statistical methods: The genetic association between an individual's genotypes at polymorphisms in positions 313 and 341 of the GSTP1 gene and weight loss was evaluated by comparing the weight loss, the BMI loss, the percentage of BMI lost between the wildtype homozygote genotype and the genotype carrying one or two variant alleles at the 5.6 month follow-up. The statistical significance of the association was assessed by using the linear regression module of the HelixTree software package (©GoldenHelix Inc Bozeman, Mont. USA).

The linear regression analysis included age and gender as covariates. The adjusted mean BMI loss, weight loss, and percentage BMI loss for each genotype was assessed using an analysis of covariance and outputting the least adjusted means in S-Plus 6. (Insightful Corp, WA). The average weight loss, BMI loss and percentage of BMI loss (as a percentage of the original BMI at baseline) for the GSTP1 polymorphism. The p-value for statistical significance refers to change in BMI as the outcome variable

TABLE 2A
Weight Loss Measures After 5.6 months - 313 alleles

| Genotype at | | | | | |
|---|---|---|---|---|---|
| position 313 of | Number of | Change in | Change in | | Statistical |
| GSTP1 | individuals | BMI in kg/m2 | weight (in kg) | % BMI change | significance |
| Common | 25 | 1.81 (±0.36) | 5.12 (±1.04) | 5.93% (±3.0%) | P < 0.006 |
| genotype (AA) | | | | | |
| Variant genotype | 16 | 3.56 (±0.46) | 9.56 (±1.33) | 14.65% (±3.9%) | |
| (AG or GG) | | | | | |

TABLE 2B
Weight Loss Measures After 5.6 months - 341 alleles

| Genotype at | | | | | |
|---|---|---|---|---|---|
| position 341 of | Number of | Change in | Change in | | Statistical |
| GSTP1 | individuals | BMI | weight (in kg) | % BMI change | significance |
| Common | 24 | 1.88 (±0.37) | 5.30 (±1.08) | 5.93% (±3.0%) | P < 0.011 |
| genotype (CC) | | | | | |
| Variant | 15 | 3.68 (±0.47) | 9.88 (±1.38) | 15.2% (±4.1%) | |
| genotype (CT or | | | | | |
| TT) | | | | | |

### Example 2

This example illustrates the genetic association between GSPT1 polymorphoism and body mass index. Genotype, BMI and nutrient intake data was collected from 3000 customers who have taken the Sciona MyCellf™ nutrigenetics test (all data was anonymized before analysis) whose self reported ethnicity was White. Genotyping and nutrient intake analysis was carried out as described in Example 1. Gene-Nutrient-BMI interactions were analyzed in individual self-declared ethnic groups, and by gender.

The genetic association between an individual's genotypes at polymorphisms in positions 341 of the GSTP1 gene and weight loss was evaluated by comparing BMI between the wildtype homozygote genotype and the genotype carrying one or two variant alleles. The statistical significance of the association was assessed by using the linear regression module of the HelixTree software package (©GoldenHelix Inc Bozeman, Mont. USA). The linear regression analysis included age and gender as covariates. Individuals carrying one or two copies of the variant allele (“T”) have lower body mass index than those with the wildtype genotype, as shown in Table 3.

TABLE 3
BMI of CC vs CT/TT alleles

| Genotype at position 341 of | Number of | | Statistical |
|---|---|---|---|
| GSTP1 | individuals | BMI | significance |
| Common genotype (CC) | 2549 | 27.18 (±0.12) | P < 0.036 |
| Variant genotype | 456 | 26.45 (±0.28) | |
| (CT or TT) | | | |

### Example 3

In this and the following examples the term “gene-by-environment interaction” or “gene-by-nutrient interaction” refers to the situation where the effect on a trait, for example body mass index, is only seen under some environmental (such as could be the age of the individual) or nutrient intake conditions or where a different effect on the trait is seen depending on the nutrient intake or environmental conditions.

This example illustrates the differential effect on genotype at GSTP1 polymorphism at position 313 on body mass index in regards to vitamin A intake.

### Statistical Assessment of Gene by Nutrient Interaction Using Linear Regression Models or Analyses of Variance:

In statistics, an interaction is a term in a statistical model added when the effect of two or more variables is not simply additive. Such a term reflects that the effect of one variable depends on the values of one or more other variables. In the present case BMI=aX1+bX2+e where X1 can represent the value of an individual's intake of a given nutrient or an individual's age and X2 can represent an individual's genotype, a and b are coefficients to be estimated by the regression model. In contrast to this, BMI=aX1+bX2+c (X1×X2)+e is an example of a model with an interaction between variables X1 and X2 (“e” refers to the random variable whose value is the amount by which the observed BMI differs from the expected value of BMI given the linear model fitted). The interacting variables can be categorical variables (e.g. genotype) or real numbers. The consequence of an interaction is that the effect of one variable depends on the value of another.

An analysis of variance or a multiple linear regression model will produce values indicating the probability that the difference is due to their being an actual difference (as opposed to the difference being the result of coincidence) for the genotype variable, the nutrient variable and the interaction variable. Significance values will be obtained for each individual factor (ie main effects) as well as the significance of factor interrelationships (i.e. interaction effects). With an interaction effect, a significant p-value is returned when two or more factors are considered. If an interrelationship between the factors (nutrient intake and genotype or age and genotype) is found and ANOVA or a linear regression model will yield a statistically significance probability value (e.g. <0.05). (James J. Jaccard, Robert Turrisi, Interaction Effects in Multiple Regression, Sage

Publications, 2003, ISBN 0-7619-2742-5). The statistical significance of gene by nutrient interactions or gene by age interactions have been estimated by including an interaction term in linear regression model in S-Plus 6.0.

The genetic association between GSTP1 genotype at position 313 was studied separately among individuals in the bottom half of the vitamin A intake (less than 11,000 IU/day) and those in the top half of vitamin A intake (11,000 IU/day or above).

TABLE 4A
Subjects in the lower 50% of vitamin A intake

| GSTP1 313 genotype | AA | AG | GG |
|---|---|---|---|
| BMI | 27.11 | 27.72 | 27.996 |
| Std err | 0.222 | 0.228 | 0.454 |

Genotype at position 313 is significantly associated with BMI among this group of people with p<0.025 as found by analysis of variance which included genotype as a categorical variable 0, 1, 2 depending on the number of copies of the “G” allele carried at this polymorphism and age and gender as covariates.

TABLE 4B
Subjects in the top 50% of vitamin A intake:

| GSTP1 313 genotype | AA | AG | GG |
|---|---|---|---|
| BMI | 26.774 | 26.677 | 26.013 |
| se | 0.212 | 0.213 | 0.413 |

Genotype at position 313 is not significantly associated with BMI among this group of people with p<0.16 as found by analysis of variance which included genotype as a categorical variable 0, 1, 2 depending on the number of copies of the “G” allele carried at this polymorphism and age and gender as covariates.

A linear regression model fitted using the software S-Plus 6.0 which included GSTP1 genotype at position 313 (GSTP1_{—}313 for short), vitamin A intake, age, gender and the interaction of vitamin A and GSTP1 genotype showed that the interaction term is statistically significant with p<0.01. The main effect of GSTP1_{—}313 on BMI was not statistically significant and the main effect of vitamin A was statistically significant with p<0.0001.

Thus, a significant association with higher BMI of the G allele on BMI is seen only among individuals who have a low intake of vitamin A.

In the case of the 313 alleles, the beneficial effect of the variant AG or GG genotype is not observed in subjects with low vitamin A intake. Without sufficient vitamin A intake, the AG and GG genotypes is associated with an increased BMI. This effect is negated by sufficient vitamin A intake in the diet.

### Example 4

This example illustrates the differential effect of on genotype at GSTP1 polymorphism at position 341 on body mass index depending on cruciferous vegetable intake. The genetic association between GSTP1 genotype at position 341 was studied separately among individuals in the bottom half of cruciferous vegetables intake (less than 3 servings per week) and those in the top half of cruciferous vegetables intake (more than 3 servings per week).

TABLE 5A
Individuals in the top 50% of cruciferous vegetables intake

| GSPT1 341 genotype | CC | CT or TT |
|---|---|---|
| BMI | 26.47 | 25.315 |
| Std err | 0.229 | 0.504 |

The genotype at position 341 was significantly associated with BMI among this group of people with p<0.036 as found by analysis of variance which included genotype as a categorical variable 0, 1 depending on whether an individual carried the T allele (1) or not (0) Age and gender were included as covariates.

TABLE 5B
Individuals in the bottom 50% of cruciferous vegetables intake:

| GSPT1 341 genotype | CC | CT or TT |
|---|---|---|
| BMI | 27.322 | 27.098 |
| Std err | 0.13 | 0.314 |

Genotype at position 341 is not significantly associated with BMI among this group of people with p<0.43 by analysis of variance.

A linear regression model fitted using the software S-Plus 6.0 which included GSTP1 genotype at position 341 (GSTP1_{—}341 for short), cruciferous vegetables intake, age, gender and the interaction of cruciferous vegetables and GSTP1_{—}341 genotype showed that the interaction term is statistically significant with p<0.02. Thus, a significant association with lower BMI of the T allele on BMI is seen preferentially among individuals who have a large intake of cruciferous vegetables.

The beneficial effect of the CT or TT 341 genotype was observed in individuals with a high cruciferous vegetable intake, the benefit was not seen in those with a low intake of this food subtype.

### Example 5

This example illustrate the differential effect of on genotype at GSTP1 polymorphism at position 313 on body mass index depending on calcium intake. This was determined in an analogous manner to Examples 3 and 4 above, with the following differences being observed in low (less than 770 mg/day) and high intake (770 mg/day or above) groups.

TABLE 6A
Individuals in the Lower 50% of calcium intake

| GSTP1 313 | AA | AG | GG |
|---|---|---|---|
| BMI | 26.855 | 27.364 | 27.210 |
| Std err | 0.168 | 0.172 | 0.345 |

Association Adjusted for Age and Gender in this Group=p<0.083

TABLE 6B
Individuals in the highest 50% of calcium intake

| GSTP1 313 | AA | AG | GG |
|---|---|---|---|
| BMI | 27.376 | 26.536 | 26.352 |
| Std err | 0.383 | 0.381 | 0.684 |

Association with BMI Adjusted for Age and Gender p<0.084
Interaction Between Calcium Intake and GSTP1_{—}313 Genotype p<0.027.

The wildtype (AA) genotype is associated with higher BMI in individuals that have a high intake of calcium but with lower BMI among individuals with a low intake of calcium.

The 313 genotype has an effect on BMI in relation to calcium intake. In particular, those individuals with an AA genotype appear to benefit (in terms of lower BMI) from a lower calcium intake, whereas a high calcium intake appears to be detrimental to such a genotype. Conversely, those with a G allele at 313 (i.e. AG and GG genotypes) appear to benefit from a high intake of calcium.

### Example 6

The term “gene-by-environment interaction” or “gene-by-nutrient interaction” refers to the situation where the effect on a trait, for example body mass index, is only seen under some environmental (such as could be the age of the individual) or nutrient intake conditions or where a different effect on the trait is seen depending on the nutrient intake or environmental conditions.

This example illustrates the differential effect of at IL-6 polymorphism at position −174 on body mass index depending on age. The genetic association between IL6 genotype at position −174 was studied separately among individuals aged less than 50 years of age and those aged 50 and older.

TABLE 7A
Individuals aged under 50 years of age

| IL6-174 genotype | CC | CG | GG |
|---|---|---|---|
| BMI | 26.072 | 26.439 | 26.999 |
| Std err | 0.346 | 0.214 | 0.235 |

By ANOVA the genotype at position −174 of the IL6 gene was found to be significantly associated with BMI among these individuals under the age of 50, (p<0.016) with the G allele being associated with higher BMI (Table 7A). The analysis of variance which included genotype as a categorical variable 0, 1 or 2 depending on the number of copies of the C (variant) allele. Gender was included as a covariate.

TABLE 7B
Individuals aged 50 or more years of age

| IL6-174 genotype | CC | CG | GG |
|---|---|---|---|
| BMI | 28.153 | 27.276 | 27.649 |
| se | 0.353 | 0.219 | 0.242 |

By ANOVA the genotype at position −174 of the IL6 gene was not associated with BMI among this individuals aged 50 or older, (p<0.49) although in these age group individuals carrying the G allele have lower BMI (Table 7B).

A linear regression model fitted using the software S-Plus 6.0 which included IL6 genotype at position −174, age, gender and the interaction of age and IL6 −174 genotype showed that the interaction term was not significant, with a p value of p<0.086.

The foregoing description of the present invention has been presented for purposes of illustration. The description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art. Each publication and reference cited herein is incorporated herein by reference in its entirety.

What is claimed is:

1. A method for selecting a weight loss program for an individual comprising
a. determining the individual's GSTP1 genotype at loci position 313, 341 or both;
b. selecting a weight loss program for the individual when the individual comprises a genotype selected from the group consisting of homozygous for the G allele at position 313, heterozygous (A/G) at position 313, homozygous for the T allele at position 341, heterozygous (C/T) at position 341, and combinations thereof wherein the weight loss program is modified from a weight loss program for a comparable individual who is wild-type at positions 313 and 341.

2. The method of claim 1, wherein the genotype of the individual comprises either homozygous for the G allele at position 313 or heterozygous (A/G) at position 313, and wherein the individual is predicted to obtain a greater response to the weight loss program compared to an individual homozygous for the A allele at position 313.

3. The method of claim 1, wherein the genotype of the individual comprises either homozygous for the T allele at position 341 or heterozygous (C/T) at position 341, and wherein the individual is predicted to obtain a greater response to the weight loss program compared to an individual homozygous for the C allele at position 341.

4. The method of claim 1, wherein the genotype of the individual comprises either homozygous for the G allele at position 313 or heterozygous (A/G) at position 313, and wherein the selected weight loss program comprises a dietary program higher in energy intake or shorter in duration compared to a weight loss program for a comparable individual having a wild-type genotype at position 313.

5. The method of claim 4, wherein the selected weight loss program comprises a higher calcium intake compared to a weight loss program for a comparable individual having a wild-type genotype at position 313.

6. The method of claim 1, wherein the genotype of the individual comprises homozygous for the C allele at position 341, wherein the weight loss program comprises a dietary program lower in energy intake or longer in duration compared to an individual having a genotype consisting of a T allele at position 341.

7. The method of claim 1, wherein the genotype of the individual comprises homozygous for the A allele at position 313 and/or homozygous for the C allele at position 341, wherein the weight loss program comprises a dietary program lower in energy intake or longer in duration compared to an individual having a genotype comprising homozygous for the G allele at position 313 or heterozygous (A/G) at position 313 and comprising homozygous for the T allele at position 341 or heterozygous (C/T) at position 341.

8. The method of claim 1, wherein the genotype of the individual comprises either homozygous for the T allele at position 341 or heterozygous (C/T) at position 341, and wherein the selected weight loss program comprises a dietary program higher in cruciferous vegetable intake compared to a weight loss program for a comparable individual having a wild-type genotype at position 341.

9. The method of claim 1, wherein the genotype of the individual comprises either homozygous for the G allele at position 313 or heterozygous (A/G) at position 313, and wherein the selected weight loss program comprises a dietary program with higher vitamin A intake compared to a weight loss program for a comparable individual having a wild-type genotype at position 313.

10. The method of claim 1, wherein the genotype of the individual comprises either homozygous for the G allele at position 313 or heterozygous (A/G) at position 313, and wherein the selected weight loss program comprises a dietary program with higher calcium intake compared to a weight loss program for a comparable individual having a wild-type genotype at position 313.

11. The method of claim 1, wherein the genotype of the individual comprises either homozygous for the G allele at position 313 or heterozygous (A/G) at position 313 and comprises either homozygous for the T allele at position 341 or heterozygous (C/T) at position 341, and wherein the selected weight loss program comprises a dietary program with higher vitamin A intake compared to a weight loss program for a comparable individual having a wild-type genotype at position 313 and wherein the selected weight loss program comprises a dietary program with higher cruciferous vegetable intake compared to a weight loss program for a comparable individual having a wild-type genotype at position 341.

12. The method of claim 1, wherein the genotype of the individual comprises a genotype of homozygous for the G allele at position 313 or heterozygous (A/G) at position 313 and comprises homozygous for the T allele at position 341 or heterozygous (C/T) at position 341, and wherein the selected weight loss program comprises a dietary program with higher calcium intake compared to a weight loss program for a comparable individual having a wild-type genotype at position 313 and comprises a dietary program with higher cruciferous vegetable intake compared to a weight loss program for a comparable individual having a wild-type genotype at position 341.

13. A method for selecting a dietary plan for an individual in need of achieving an increase in body weight, the method comprising
a. determining the individual's GSTP1 genotype at loci position 313 of the GSTP1 gene;
b. selecting a dietary program for the individual when the individual comprises a genotype comprising homozygous for the A allele at position 313, wherein the dietary program recommends a lower calcium intake compared to a dietary program for an individual who is not in need of achieving an increase in body weight.

14. The method of claim 1, wherein the GSTP1 allele is determined as part of a panel of at least 5 genes that have one or more alleles wherein the other genes are selected from methylene-metra-hydro-folate-reductase (MTHFR); metyhionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein C-III (APOC3); apolipoprotein A-V (APOA5); cholesteryl ester transfer protein (CETP); ipoprotein lipase (LPL); endothelial nitric oxide synthase (eNOS); angiotensin converting enzyme gene (ACE); vitamin D receptor (VDR); collagen type I alpha 1 (COL1A1); tumor necrosis factor alpha (TNF-α); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

15. A method for selecting a weight loss program for an individual under the age of 50, comprising
a. determining the individual's IL-6 genotype at loci position −174;
b. selecting a weight loss program for the individual when the individual comprises a genotype selected from the group consisting of homozygous for the G allele at position −174 and heterozygous (C/G) at position −174, wherein the weight loss program is modified from a weight loss program for a comparable individual who is homozygous for the C allele at position −174.

16. The method of claim 15, wherein the individual's genotype is homozygous for the C allele at position −174, wherein the individual is predicted to obtain a greater response to a weight loss program compared to an individual having a genotype selected from the group consisting of homozygous for the G allele at position −174 and heterozygous (C/G) at position −174.

17. The method of claim 15, wherein the individual's genotype is selected from the group consisting of homozygous for the G allele at position −174 and heterozygous (C/G) at position −174, and wherein the selected weight loss program comprises a dietary program lower in energy intake and/or longer in duration compared to an individual comprising a genotype of homozygous for the C allele at position −174.

18. The method of claim 15, wherein the IL-6 allele is determined at part of a panel of at least 5 genes that have one or more alleles wherein the other genes are selected from methylene-metra-hydro-folate-reductase (MTHFR); metyhionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein C-III (APOC3); apolipoprotein A-V (APOA5); cholesteryl ester transfer protein (CETP); ipoprotein lipase (LPL); endothelial nitric oxide synthase (eNOS); angiotensin converting enzyme gene (ACE); vitamin D receptor (VDR); collagen type I alpha 1 (COL1A1); tumor necrosis factor alpha (TNF-α); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

19. The method of claim 13, wherein the GSTP1 allele is determined as part of a panel of at least 5 genes that have one or more alleles wherein the other genes are selected from methylene-metra-hydro-folate-reductase (MTHFR); metyhionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein C-III (APOC3); apolipoprotein A-V (APOA5); cholesteryl ester transfer protein (CETP); ipoprotein lipase (LPL); endothelial nitric oxide synthase (eNOS); angiotensin converting enzyme gene (ACE); vitamin D receptor (VDR); collagen type I alpha 1 (COL1A1); tumor necrosis factor alpha (TNF-α); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).


SEQUENCE LISTING (11 sequences)

SEQ ID NO: 1 - DNA, 633 bp, Homo sapiens
  misc_feature: GSTP1 coding
atgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagcgcccggcccaagctcaaggccttcctggcctcccctgagtacgtgaacctccccatcaatggcaacgggaaacagtga

SEQ ID NO: 2 - protein, 210 aa, Homo sapiens
  misc_feature: GSTP1
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 3 - DNA, 986 bp, Homo sapiens
  misc_feature: GSTP1
tgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccatgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagctaaaaaaaaaaaaaaaaaaaaaaaaaaaaa

SEQ ID NO: 4 - DNA, 708 bp, Homo sapiens
  allele (313)..(313): rs1695; r at position 313 may be either a or g
atgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacrtctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagcgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagct

SEQ ID NO: 5 - protein, 210 aa, Homo sapiens
  misc_feature: Variant 313
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYXSLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 6 - DNA, 708 bp, Homo sapiens
  misc_feature: allele 341
atgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggsgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagcgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagct

SEQ ID NO: 7 - protein, 210 aa, Homo sapiens
  MISC_FEATURE: Variant 341
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEXGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 8 - DNA, 601 bp, Homo sapiens
  misc_feature: allele IL-6 -174
actcagttcagaacatctttggtttttacaaatacaaattaactggaacgctaaattctagcctgttaatctggtcactgaaaaaaaattttttttttttcaaaaaacatagctttagcttattttttttctctttgtaaaacttcgtgcatgacttcagctttactctttgtcaagacatgccaaagtgctgagtcactaataaaagaaaaaaagaaagtaaaggaagagtggttctgcttcttagcgctagcctcaatgacgacctaagctgcacttttccccctagttgtgtcttgcsatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctcattctgccctcgagcccaccgggaacgaaagagaagctctatctcccctccaggagcccagctatgaactccttctccacaagtaagtgcaggaaatccttagccctggaactgccagcggcggtcg

SEQ ID NO: 9 - DNA, 52 bp, Homo sapiens
  misc_feature: allele rs1800795
cacttttccccctagttgtgtcttgcratgctaaaggacgtcacattgcaca

SEQ ID NO: 10 - DNA, 1201 bp, Homo sapiens
  misc_feature: IL-6
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 11 - protein, 212 aa, Homo sapiens
  misc_feature: IL-6
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM